(19) United States
(12) Patent Application Publication
(10) Pub. No.: US 2022/0218781 A1
(43) Pub. Date: Jul. 14, 2022

(54) COMPOSITION FOR ENHANCEMENT OF BIOAVAILABILITY OF PHYTOCHEMICALS AND PROCESS FOR PREPARATION THEREOF

(71) Applicant: OLENE LIFE SCIENCES PRIVATE LIMITED, Chennai (IN)

(72) Inventors: Somashekara NIRVANASHETTY, Chennai (IN); Sanjib Kumar PANDA, Chennai (IN); Vivek Anand PARACHUR, Chennai (IN)

(21) Appl. No.: 17/554,636

(22) Filed: Dec. 17, 2021

Related U.S. Application Data
(63) Continuation-in-part of application No. PCT/IN2020/050545, filed on Jun. 21, 2020.

(30) Foreign Application Priority Data
Jun. 21, 2019 (IN) 201941024774

(51) Int. Cl. A61K 36/9068; A61K 36/9066; A61K 36/21; A61K 36/81; A61K 36/886; A61K 36/23; A61K 36/22; A61K 36/47; A61K 36/889; A61K 36/8962; A61K 36/42; A61K 31/047; A61K 31/122; A61K 31/4525; A61K 31/19; A61K 31/164; A61K 31/198; A61K 31/357; A61K 31/4375; A61K 31/136; A61K 36/534; A61K 31/593

(52) U.S. Cl. CPC A61K 36/9068; A61K 2236/37; A61K 36/21; A61K 36/81; A61K 36/886; A61K 36/23; A61K 36/22; A61K 36/47; A61K 36/889; A61K 36/8962; A61K 36/42; A61K 31/047; A61K 31/122; A61K 31/4525; A61K 31/19; A61K 31/164; A61K 31/198; A61K 31/357; A61K 31/4375; A61K 31/136; A61K 36/534; A61K 31/593; A61K 2236/31; A61K 36/9066

(57) ABSTRACT

A composition for enhancing the self-dispersion and bioavailability of phytochemicals includes (i) phytochemicals selected from carotenoids, stilbenoid, isoflavones, terpenes, isoquinoline alkaloids, phenolic compounds, saponins, flavonoids, quinones, fatty acids and their derivatives; and (ii) bio-enhancing agents/self-dispersing agents derived from extract of fresh plant parts selected from Curcuma longa (Turmeric); Beta vulgaris (Beetroot), Solanum tuberosum (Potato), Aloe barbadensis (Aloe vera), Daucus carota (Carrot), Mangifera indica (Mango), Manihot esculenta (Tapioca) and Cocos nucifera (Coconut) and bulb of Allium sativum (garlic). A process for preparation of the composition is also disclosed herein.

### CROSS-REFERENCE TO RELATED APPLICATIONS

This application is a continuation-in-part of parent International Application No. PCT/IN2020/050545, filed on Jun. 21, 2020, which claims priority to Indian Patent Application No. 201941024774, filed on Jun. 21, 2019. The entire disclosure of each prior application is incorporated by reference herein in its entirety.

### TECHNICAL FIELD OF THE INVENTION

The present invention relates to a composition comprising phytochemicals and bio-enhancing agents/self-dispersing agents derived from fresh plant parts, wherein said composition enhances the self-dispersion and bioavailability of phytochemicals. The present invention also relates to a process for preparation of said composition.

### BACKGROUND OF THE INVENTION

Herbal medicine has made a major contribution to the drug discovery process and its use has increased worldwide due to their therapeutic effects and fewer adverse effects as compared to the modern medicines. Many herbal drugs and herbal extracts, however, despite their effective in-vitro findings demonstrate less or negligible in-vivo activity due to their poor solubility/dispersibility resulting in poor absorption and hence poor bioavailability leading to poor or decreased efficacy.

The phytochemicals with poor bioavailability in plant extracts belong to category of phenolic compounds, xanthophyllic carotenoids, carotenoids, protoberberine group of benzylisoquinoline alkaloids, stilbenoids, isoflavones, monoamine alkaloids, flavonols and many others which have proven to have several therapeutic benefits like anti-inflammatory, anti-oxidant, age-related macular degeneration, anti-cancerous, memory enhancing among many other medicinal activities. However, very less has been achieved with respect to said phytochemicals for prevention and treatment of diseases due to their poor intestinal absorption and bioavailability.

The further developments in the art observed that the main issue which needs to be addressed with the phytochemicals is not only the bioavailability but also their availability in the systemic circulation for longer period of time in biologically active form for sustained efficacy. This will also help in reducing the dosages thus making the treatment cost effective, minimize drug toxicity and is patient compliant.

Bioavailability enhancing activity of natural compounds have several mechanisms of action and is mainly attributed to mechanisms such as P-glycoprotein (P-gp) inhibition activity, non-specific mechanisms promoting rapid absorption of drugs such as increased blood supply to the gastrointestinal tract, decreased hydrochloric acid secretion, non-specific mechanisms inhibiting enzymes participating in metabolism of drugs to list the few.

‘Bioavailability enhancers’ are facilitators capable of increasing the intestinal absorption, enhancing bioavailability and bio-efficacy of a particular drug or phytochemical with which it is combined, without any typical pharmacological activity of its own at the dose used.

The term ‘bioavailability enhancer’ was first coined by Indian scientists at the Regional Research Laboratory, Jammu (RRL, now known as Indian Institute of Integrative Medicine, Jammu), who discovered and scientifically validated piperine as the world's first bioavailability enhancer in 1979 [Atal C K. A breakthrough in drug bioavailability-a clue from age old wisdom of Ayurveda. IDMA Bulletin. 1979; 10:483-484].

The use of herbal bio-enhancers is widely appreciated as a means for bioavailability enhancement because these are safe, non-toxic, economical, easily procured, pharmacologically inert and non-allergenic in nature.

WO03/049753 relates to a bio-enhancing composition containing extract and/or bioactive fraction/isolate from the plant Zingiber officinale in combination with drugs, nutrients, nutraceuticals, micronutrients and herbal drugs/products and optionally containing piperine as a extract/active fraction (solvent extract) obtained from Piper nigrum, Piper longum or its oleoresin as a bioavailability enhancer and its process for producing the extract or fractions from the plant source.

U.S. Pat. No. 5,536,506 discloses use of piperine to increase the bioavailability of nutritional compounds. US'506 further discloses a composition and method for the improvement of gastrointestinal absorption and systemic utilization of nutrients and nutritional supplements, wherein the composition comprises an solvent extract from the fruit of Piper containing a minimum of 98% of pure alkaloid piperine.

WO2015/025263 discloses a composition containing curcumin along with soluble proteins, dietary fiber, fixed oil and essential oil from turmeric that increases absorption of curcumin in the blood and results in better dispersibility upon oral consumption, which is useful for treatment of various diseases.

Article titled, “Bioavailability enhancing activities of natural compounds from medicinal plants” by Myung Joo Kang et. al., published in Journal of Medicinal Plants Research Vol. 3(13), pp. 1204-1211, December, 2009 disclose the natural compounds such as quercetin, genistein, naringin, sinomenine, piperine, glycyrrhizin and nitrile glycoside for improving the drug bioavailability.

Though bio-enhancers in drug delivery are known, challenge still persists to provide safe herbal bio-enhancers for increasing the bioavailability of herbal origin/phytochemicals that are non-toxic, non-irritating, rapid acting with predictable activity. Natural bioenhancers produced using solvent free process are preferred over the bioenhancers produced using solvent extraction due to the adverse effect of residual solvents. Another problem encountered in the art is large scale production of such formulations containing the herbal compound and the bio-enhancer.

### OBJECT OF THE INVENTION

In view of the shortcomings of the art, the object of the present invention is to provide a potential solution for enhancing the bio-availability of phytochemicals using bio-enhancing/self-dispersing agents derived from solvent free extract of fresh plant parts.

### SUMMARY OF THE INVENTION

To achieve the above objective, in an aspect, the present invention provides a composition for enhancing the self-dispersion, intestinal absorption and bioavailability of phytochemicals comprising;
a) phytochemicals selected from the group consisting of carotenoids, stilbenoid, isoflavones, sterols, terpenes, isoquinoline alkaloids, phenolic compounds, saponins, flavonoids, quinones, fatty acids and their derivatives either alone or mixtures thereof; and
b) bio-enhancing agents/self-dispersing agents derived from extract of fresh plant parts such as fruits, seeds leaves, and rhizomes selected from Turmeric, Ginger, Beetroot, berry fruits, Allium sativum bulb, Potato, Aloe vera, Carrot, Mango, Monk fruit, Tapioca, fenugreek seeds and Coconut either alone or mixtures thereof.

In another aspect, the present invention provides a process for preparation of the composition for enhancing the self-dispersion, intestinal absorption and bioavailability of phytochemicals comprises the steps of;
a) Cleaning and slicing the fresh plant parts selected from turmeric, beet root, potato, Aloe vera, carrot, mango, berries, monk fruit, fenugreek seeds, tapioca and coconut either alone or mixtures thereof into small pieces and extracting the fresh liquid extract from them to obtain the bio-enhancing agents/self-dispersing agents; followed by filtering the liquid extract to remove sedimentable particles;
b) homogenizing the mixture of step (a) for 30 minutes between 25° C. to 80° C. at 200 to 10000 RPM; and
c) adding phytochemicals selected from the group consisting of carotenoids, stilbenoid, isoflavones, terpenes, isoquinoline alkaloids, phenolic compounds, saponins, flavonoids, quinones, fatty acids and their derivatives to the liquid extract of step (a);
d) homogenizing the mixture of step (c) for 30 to 60 minutes between 25° C. to 80° C. at 200 to 10000 RPM to obtain slurry; and
e) vacuum drying the slurry of step (d) at 25° C. to 85° C. for 8 hours followed by milling the flakes to obtain free flowing powder (particle size>120 mesh) of the composition.

### BRIEF DESCRIPTION OF THE FIGURES

FIG. 1: Release profile of Lutein composition of Example 1 Vs. unformulated lutein extract

FIGS. 2A and 2B: Comparative self-dispersion profile of Lutein extract composition of Example 1 (FIG. 2A) vis-à-vis Standard unformulated lutein extract (FIG. 2B).

FIGS. 3A and 3B: Comparative self-dispersion profile of Boswellia extract composition of Example 13 (FIG. 3A) vis-à-vis Standard unformulated Boswellia extract (FIG. 3B).

FIG. 4: Release profile of Pepper extract Composition of Example 16 Vs. Standard unformulated pepper extract (95% Piperine).

FIGS. 5A and 5B: Comparative self-dispersion profile of Pepper extract composition of Example 16 (FIG. 5A) vis-à-vis Standard unformulated pepper extract (FIG. 5B).

FIGS. 6A and 6B: Comparative self-dispersion profile of Coenzyme Q10 Composition of Example 22 (FIG. 6A) vis-à-vis Standard unformulated Coenzyme Q10 extract (FIG. 6B).

FIGS. 7A and 7B: Comparative self-dispersion profile of Palmitoylethanolamide Composition of Example 25 (FIG. 7A) vis-à-vis Standard unformulated Palmitoylethanolamide (FIG. 7B).

### DETAILED DESCRIPTION OF THE INVENTION

The present invention will now be described in detail in connection with certain embodiments, so that various aspects thereof may be fully understood and appreciated.

Source of Phytochemicals Used in the Invention:

The phytochemicals used in the invention are procured from the vendors. The details of which are provided as follows:

Lutein: Sourced from Plant Lipids Private Limited Kolenchery, Cochin—682 311, Kerala, India.

Lycopene: Sourced from SV Agrofood, India C-9/150, Yamuna Vihar, Delhi—53, India.

Astaxanthin: Sourced from Bio-gen Extracts Pvt. Ltd. Plot No. 57, 1st Stage, Sompura Industrial Area, Dobaspet, Bangalore—562 111, Karnataka, India.

Piperine: Sourced from Plant Lipids Private Limited Kolenchery, Cochin—682 311, Kerala, India

Boswellia: Sourced from Sami Labs Limited, 19/1, 1^{st} Main Rd, 2^{nd} Phase, Nalagadderanahalli, Peenya, Bengaluru, Karnataka—560058, India.

Beta-carotene: Sourced from SV Agrofood, India C-9/150, Yamuna Vihar, Delhi—53, India.

Berberine: Sourced from Kuber Impex Ltd 304-5/The Magnate tower 16/1 New Palasia Indore Madhya Pradesh—452001, India.

Resveratrol: Sourced from Kuber Impex Ltd 304-5/The Magnate tower 16/1 New Palasia Indore Madhya Pradesh-452001, India.

Genistein: Sourced from Kuber Impex Ltd 304-5/The Magnate tower 16/1 New Palasia Indore Madhya Pradesh-452001, India.

Coenzyme Q10: Sourced from SV Agrofood, India C-9/150, Yaruna Vihar, Delhi—53, India.

Palmitoylethanolamide (PEA): Sourced from Wuxi Cima Science Co. Ltd, 288, Shi Ba Wan Road, Wuxi 214064, Jiangsu, China.

Vitamin D: Sourced from Supreme Pharmaceuticals Mysore Pvt. Ltd. #73, 74 & 48 P-1 KIADB Industrial Area, Nanjangud—571302, India.

Source and Geographical Origin of the Biological Material (Plants) Used in the Invention:

Curcuma longa (Turmeric)

Geographical Origin: Native to the Indian subcontinent and Southeast Asia

Sourced from local vendor of Tamil Nadu, India

Beta vulgaris (Beetroot)

Geographical Origin: Native to the seacoasts of the Mediterranean and Europe

Sourced from local vendor of Tamil Nadu, India

Solanum tuberosum (Potato)

Geographical Origin: Native to southern Peru and extreme northwestern Bolivia

Sourced from local vendor of Tamil Nadu, India

Aloe barbadensis (Aloe vera)

Geographical Origin: Native to Arabian Peninsula

Sourced from local vendor of Tamil Nadu, India

Daucus carota (Carrot)

Geographical Origin: Native to the Southwestern Asia and Europe.

Sourced from local vendor of Tamil Nadu, India

Mangifera indica (Mango)

Geographical Origin: Native to the India, Pakistan and South Asia

Sourced from local vendor of Tamil Nadu, India

Manihot esculenta (Tapioca)

Geographical Origin: Native to the Asia, Africa, North and central west region of Brazil, South America.

Sourced from local vendor of Tamil Nadu, India

Cocos nucifera (Coconut)

Geographical Origin: Native to the Indian subcontinent and Southeast Asia.

Sourced from local vendor of Tamil Nadu, India

Allium sativum (Garlic)

Geographical Origin: Native to the Central Asia and northeastern Iran

Sourced from local vendor of Tamil Nadu, India

The term ‘phytochemical’ refers herein is as any of the various biologically active compound found in plants.

The term ‘bio-enhancing agent’ or ‘self-dispersible agent’ refers herein is an extract derived from fresh plant parts that spontaneously disperse the phytochemical(s) into an aqueous phase to form uniform suspension/solution without any external force applied to make it disperse and enhances the bio-availability of phytochemical in the composition.

The term “fresh,” as applied to a plant or plant part, may in various embodiments refer to a freshly harvested plant part, or to a plant part obtained from a freshly harvested plant. The term “fresh,” as applied to a plant or plant part, may in various embodiments refer to a plant or plant part having a water content of 40% to 98%, 65% to 98%, 65% to 97%, 70% to 97%, 75% to 97%, 78% to 96%, 80% to 96%, 85% to 95%, or 85% to 93% by weight. The term “fresh,” as applied to a plant or plant part, may in various embodiments refer to a plant or plant part having a water content of >60% by weight. In various embodiments, a fresh plant or plant part is not subjected to further post-harvest processing such as drying, milling, and/or solvent extraction.

The fresh extract of a plant part may be considered as a liquid extract obtained by extrusion of a fresh plant part containing >40% water. The fresh extract of a plant part may be a liquid extract obtained by slow extrusion, e.g., extrusion with a low-speed screw extruder, of a fresh plant part containing >40% water. The solid content in the liquid extract of a fresh plant part may be <40%, <30%, <25%, 3% to 20%, 5% to 18%, or 5% to 13%. When a liquid extract of a fresh plant part is mixed with a phytochemical (s), the ratio of liquid fresh plant part extract to phytochemicals is measured in “v/w.” In various embodiments, the liquid extract of the fresh plant part may be dried to obtain a solid, and this solid may be combined with a phytochemicals. For example, 100 mL of a liquid extract of a fresh plant part with a solids content of 10% may be dried to produce 10 g of a solid extract of a fresh plant part. Similarly, 200 mL of a liquid extract of a fresh plant part with a solids content of 5% may be dried to produce 10 g of a solid extract of a fresh plant part. In general, when amounts of the content of an extract of a fresh plant part extract are described in terms of a dry basis or a dry weight, this means either:
Solids content of a liquid extract; or
Solid mass obtained after drying a liquid extract.

The present invention arises from the need to enhance the bioavailability of plant derived phytochemicals using natural self-dispersing agents so that phytochemicals can easily be absorbed into the systemic circulation, remains for longer period of time in biologically active form for sustained efficacy for various therapeutic, preventative and general health supplement applications in animals and human beings with higher safety profile.

Accordingly, in a preferred aspect, the present invention discloses a composition for enhancing the absorption and bioavailability of phytochemicals comprising;
a) phytochemical selected from the group consisting of carotenoids, stilbenoid, isoflavones, terpenes, isoquinoline alkaloids, phenolic compounds, saponins, flavonoids, quinones, fatty acids and their derivatives either alone or mixtures thereof; and
b) bio-enhancing agents/self-dispersing agents derived from extract of fresh plant parts selected from Curcuma longa (Turmeric), Beta vulgaris (Beetroot), Solanum tuberosum (Potato), Aloe barbadensis (Aloe vera), Daucus carota (Carrot), Mangifera indica (Mango), Manihot esculenta (Tapioca) Cocos nucifera (Coconut), Siraitia grosvenorii (monk fruit), berries and Allium sativum (Garlic) either alone or mixtures thereof.

The phytochemicals/phytochemicals derivatives/biologically active compounds consist of carotenoids such as Lutein, Zeaxanthin, Lycopene, astaxanthin, beta-carotene; isoquinoline alkaloids such as berberine; cannabinoids, stilbenoid such as resveratrol; isoflavones such as genistein; coenzyme Q10, Palmitoylethanolamide (PEA), Piperine, boswellic acids, Chlorogenic acids, Silymarin, silibinin, Catechin, Gingerols, Shogaols, ellagic acid, Quercitin, caffeine, caffeic acid derivatives, Iron, calcium, Ecdysteroids/ecdysterone, Rosavins, Salidroside, Curculigosides Omega-3-fatty acids, Echinasea, Gymneric acids, S-Allyl-Cystein, Melatonin, Forskolin, Huperzine, Hypericin, hyperforin, Phytoestrogens, Ginsenosides, Valerenic acid, Vitamin D, Vitamin E, vitamin K7, Spearmint extract and Theacrine and the like.

The preferable phytochemicals/phytochemicals derivatives includes lutein, astaxanthin, piperine, cannabinoids, boswellic acid, coenzyme Q10, palmitoylethanolamide, berberine, vitamin K7, Spearmint extract and vitamin D.

The phytochemicals in the composition of the present invention may be obtained either naturally and/or by synthetic and/or semi synthetic process; and can be either purified molecule(s) or extract(s).

The bio-enhancing agents/self-dispersing agents in the composition of the present invention are derived from the extracts of fresh plant parts selected from rhizomes of Curcuma longa (Turmeric), taproots of Beta vulgaris (Beet root), tubers of Solanum tuberosum (Potato), leaf of Aloe barbadensis (Aloe vera), taproots of Daucus carota (Carrot), fruits of Mangifera indica (Mango), roots of Manihot esculenta (Tapioca) fruits of Cocos nucifera (Coconut), fruit of Siraitia grosvenorii (monk fruit), berries and bulb of Allium sativum (garlic).

The bio-enhancing agent/self-dispersing agent enhances the bioavailability of phytochemicals through self-dispersion.

The extracts of fresh plant parts are obtained without the use of any solvents.

In another preferred embodiment, the present composition comprises phytochemicals in an amount ranging from 1% to 99%, preferably, 5% to 95% of the total composition.

In another preferred embodiment, the present composition comprises bio-enhancing agents/self-dispersing agents obtained from fresh plant parts is in an amount ranging from 5% to 95% of the total composition.

In another embodiment, the present invention discloses a method for enhancing the bioavailability of phytochemicals using bio-enhancing agent/self-dispersing agent derived from extracts of fresh plant parts; wherein said method comprises solvent free extraction of fresh plant parts followed by homogenizing the plant extracts with phytochemicals at higher temperature and drying, powdering to get the free-flowing powder of self-dispersible composition.

In another preferred embodiment, the present invention discloses a process for preparation of composition for enhancing the bioavailability of phytochemicals comprises the steps of;
a) cleaning and slicing the fresh rhizome Curcuma longa, taproot of Beta vulgaris, tuber of Solanum tuberosum, leaf of Aloe barbadensis, taproot of Daucus carota (Carrot), fruit of Mangifera indica (Mango), root of Manihot esculenta (Tapioca) and fruit of Cocos nucifera (Coconut) fruit of Siraitia grosvenorii, berries and bulb of Allium sativum (garlic) either alone or mixtures thereof into small pieces and extracting the fresh extract using low RPM screw extruder with 0.5 mm to 1 mm mesh/filter press to obtain the extract of bio-enhancing agent/self-dispersing agent;
b) filtering the extract of step (a) using 100-500 micron filter to remove sedimentable particles followed by heating the extract for 30 minutes at 25-60° C. with continuous stirring using a homogenizer;
c) adding phytochemicals selected from the group consisting of carotenoids, stilbenoid, isoflavones, terpenes, isoquinoline alkaloids, saponins, flavonoids, quinones, fatty acids and their derivatives either alone or mixtures thereof to the extract of step (b); and
d) homogenizing the mixture of step (c) at 200 to 10000 RPM for 30 to 60 minutes at 25° C. to 80° C. followed by vacuum drying at 40° C. to 85° C., and powdering to obtain free-flowing powder (particle size>120 mesh) of self-dispersible composition.

According to the process, fresh plant parts selected from rhizomes of Curcuma longa, taproot of Beta vulgaris, tubers of Solanum tuberosum, leaf of Aloe barbadensis, taproot of Daucus carota (Carrot), fruit of Mangifera indica (Mango), root of Manihot esculenta (Tapioca) fruit of Cocos nucifera (Coconut), fruit of Siraitia grosvenorii (monk fruit), berries and bulb of Allium sativum (garlic) either alone or mixtures thereof are cut into small pieces and the fresh extract was separated using low RPM screw extruder with 0.5 mm to 1 mm mesh/filter press at 20 to 35° C. The fresh plant part extract is filtered for homogenizing with phytochemicals.

The phytochemicals added to the fresh plant extract consisting of carotenoids which include Lutein, Zeaxanthin, Lycopene, astaxanthin, beta-carotene; isoquinoline alkaloids such as berberine; cannabinoids, stilbenoid such as resveratrol; isoflavones such as genistein; coenzyme Q10, Palmitoylethanolamide (PEA), Piperine, boswellic acids, Chlorogenic acids, Silymarin, silibinin, Catechin, Gingerols, Shogaols, ellagic acid, Quercitin, caffeine, caffeic acid derivatives, Iron, calcium, Ecdysteroids/ecdysterone, Rosavins, Salidroside, Curculigosides Omega-3-fatty acids, Echinasea, Gymneric acids, S-Allyl-Cystein, Melatonin, Forskolin, Huperzine, Hypericin, hyperforin, Phytoestrogens, Ginsenosides, Valerenic acid, Vitamin D, Vitamin E, Menthol and Theacrine, either alone or mixtures thereof in suitable proportion and the mixture is homogenized at 200 to 10000 RPM for about 30 to 60 minutes at 25-80° C. The mixture is then vacuum dried and powdered to obtain free-flowing powder of the present composition.

In an embodiment, the process of the present invention is solvent free.

In another embodiment, the composition of the present invention comprising one or more said phytochemicals and one or more said bio-enhancing agents/self-dispersing agent can be formulated into various dosage forms such as tablets, capsules, pills, solutions, paste, lozenges, instant mixes, ready to drink beverages (RTDs), beverages and the like.

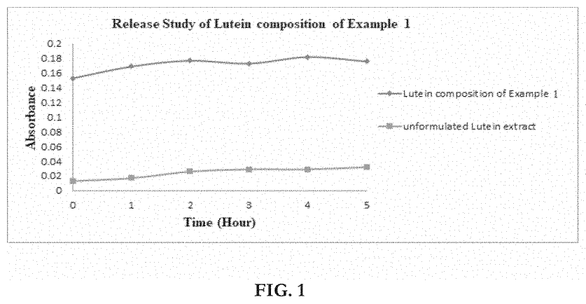

In another embodiment, the present invention discloses the release profile of lutein composition; wherein the results exhibit the increased solubility and release profile for lutein composition compared to ‘unformulated lutein extract’. The same is depicted in FIG. 1.

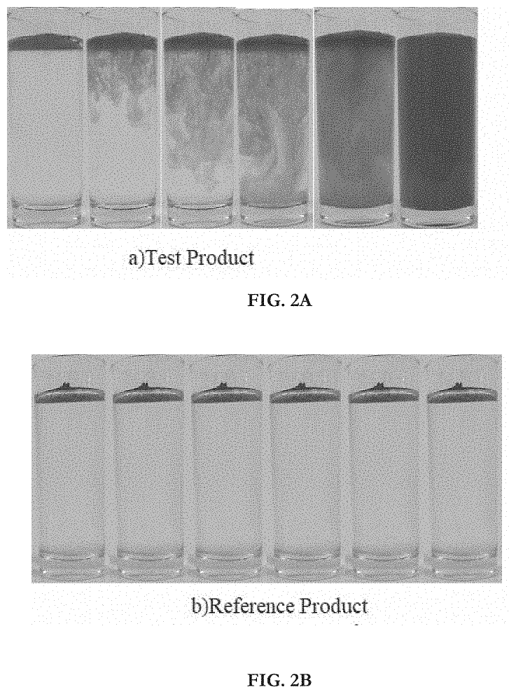

In another embodiment, the present invention discloses the comparative self-dispersion study between Lutein extract composition and standard unformulated lutein extract; wherein the result show that Lutein extract composition dispersed easily without any mechanical stirring and exhibiting enhanced self-dispersion compared to standard unformulated lutein extract which floats on top of the water. The dispersion result is given in FIG. 2.

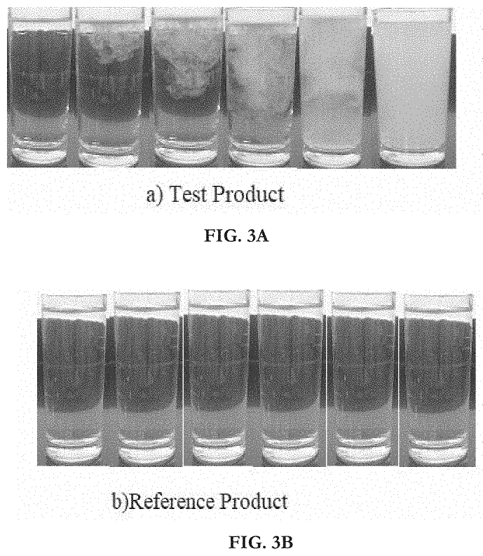

In another embodiment, the present invention discloses the comparative self-dispersion study between Boswellia extract composition and standard unformulated Boswellia extract; wherein the result show that Boswellia extract composition dispersed easily without any mechanical stirring and exhibiting enhanced self-dispersion compared to standard unformulated Boswellia extract which floats on top of the water. The dispersion result is given in FIG. 3.

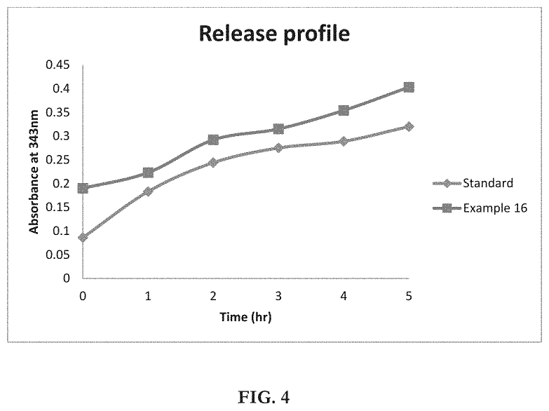

In another embodiment, the present invention discloses the release profile of pepper extract composition; wherein the results exhibit the increased solubility and release profile for pepper extract composition compared to ‘unformulated pepper extract’. The same is depicted in FIG. 4.

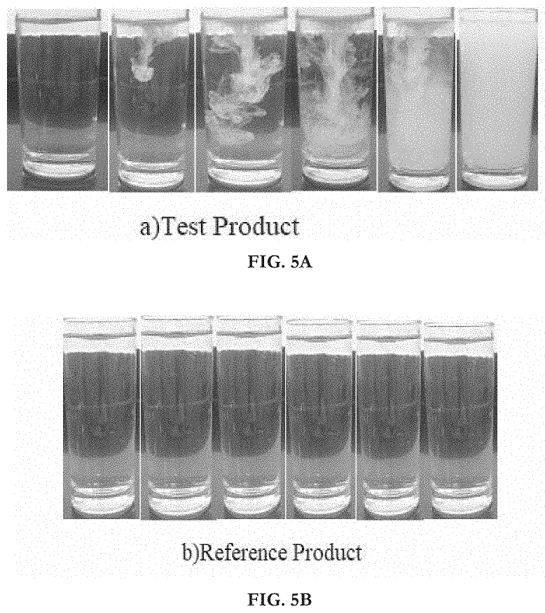

In another embodiment, the present invention discloses the comparative self-dispersion study between Pepper extract composition and standard unformulated pepper extract; wherein the result show that pepper extract composition dispersed easily without any mechanical stirring and exhibiting enhanced self-dispersion compared to standard unformulated pepper extract which floats on top of the water. The dispersion result is given in FIG. 5.

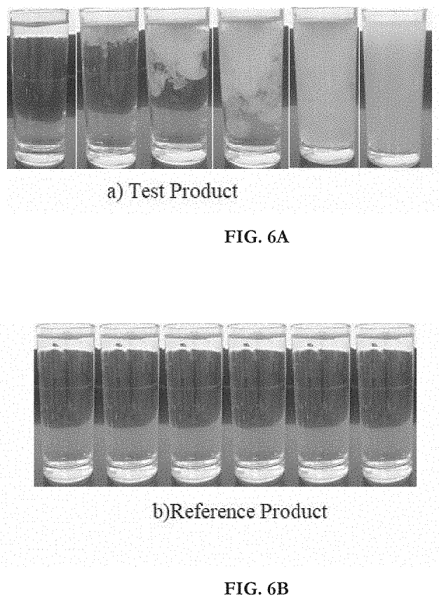

In another embodiment, the present invention discloses the comparative self-dispersion study between Coenzyme Q10 composition and standard unformulated Coenzyme Q10 extract; wherein the result show that Coenzyme Q10 composition dispersed easily without any mechanical stirring and exhibiting enhanced self-dispersion compared to standard unformulated Coenzyme Q10 extract which floats on top of the water. The dispersion result is given in FIG. 6.

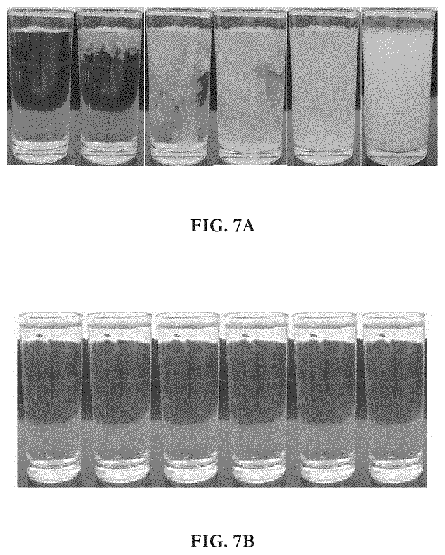

In another embodiment, the present invention discloses the comparative self-dispersion study between Palmitoylethanolamide composition and standard unformulated Palmitoylethanolamide; wherein the result show that Palmitoylethanolamide composition dispersed easily without any mechanical stirring and exhibiting enhanced self-dispersion compared to standard unformulated Palmitoylethanolamide which floats on top of the water. The dispersion result is given in FIG. 7.

In an optional embodiment, the composition of the present invention comprising anti-oxidants, wetting agents, glidants selected from ascorbic acid, silicon dioxide and the like.

In another embodiment, the present invention discloses a method of treating inflammatory diseases, respiratory diseases, heart diseases, sexual diseases, cognitive diseases, eye diseases, skin diseases, anti-aging, enhancing the endurance, enhancing the energy and stress by administering therapeutically effective amount of present composition comprising one or more phytochemical and one or more bio-enhancing agent/self-dispersing agent derived from extract of fresh plant parts to a subject in need thereof; wherein the therapeutically effective amount is 5 mg to 1000 mg/per day active ingredient equivalent.

In yet another embodiment, the present invention discloses the composition comprising one or more phytochemical and one or more bio-enhancing agent/self-dispersing agent derived from extract of fresh plant parts used in the treatment of inflammatory disease, respiratory diseases, heart diseases, sexual diseases, cognitive diseases, eye diseases, skin disease, anti-aging, enhancing the endurance, enhancing the and stress related to animal and human beings.

In yet another embodiment, the composition of the present invention comprises phytochemical and bio-enhancing agent/self-dispersing agent derived from extract of fresh plant parts, wherein said extract from fresh plant parts can be used as/act as self-dispersing agent to enhance the bioavailability of phytochemicals, minerals and vitamins such as Iron and Vitamin D.

### EXAMPLE

Some typical examples illustrating the embodiments of the present invention are provided; however, these are exemplary only and should not be regarded as limiting the elements of the present invention.

### Example 1: Bioavailable Lutein Composition

| Sr. No. | Ingredients | Quantity |
|---|---|---|
| 1. | Lutein extract (Standardized to 80%) | 25 g |
| 2. | Fresh turmeric rhizome extract (on dry basis) | 75 g |
| | Total | 100 g |

### Example 2: Process for Preparation of Bioavailable Lutein Composition of Example 1

a) Washing 1000 g of fresh turmeric rhizome with RO water, slicing the rhizome followed by extraction using low speed screw extruder with 0.5 mm to 1 mm mesh to separate the liquid extract and filtering the extract with 100-500 micron filter;
b) heating the fresh turmeric liquid extract obtained in step (a) for 30 minutes at 40° C. with continuous stirring using a homogenizer;
c) slowly adding 25 g of lutein extract to the liquid extract (75 g of fresh turmeric rhizome extract on dry basis) of step (b) with continuous stirring;
d) homogenizing the mixture of fresh turmeric liquid extract and lutein extract of step (c) at 2000 RPM at 40° C. for 30 to minutes to obtain viscous slurry;
e) vacuum drying the slurry of step (d) at 40° C. for 8 hours using Rotary Vacuum Dryer (RVD); and
f) milling the flakes of step (e) to obtain free flowing powder (particle size>120 mesh) of self-dispersible Lutein composition.

Yield of fresh turmeric liquid extract: 750 g

Curcuminoid content of fresh turmeric liquid extract: <3%

The lutein content in the final product of Example 1 was 5.45%

### Example 3: Release Profile of Lutein Composition

The composition was tested for lutein solubility and release in phosphate buffer. 400 mg of Lutein composition from Example 1 and Standard unformulated lutein extract was added into the 400 ml phosphate buffer (pH 6.8) under intermittent stirring at 37° C. Samples were drawn for up to four hours, filtered and tested for absorbance at 446 nm using UV-Vis spectrophotometer. The results show the increased solubility and release profile for lutein composition compared to ‘unformulated lutein extract’. Absorbance vs. time graph was plotted as illustrated in FIG. 1.

### Example 4: Comparative Self-Dispersion Profile of ‘Lutein Extract Composition of Example 1’ Vis-à-Vis ‘Standard Unformulated Lutein Extract’

A comparative self-dispersion study was carried out between the ‘Composition of Example 1 (Test product)’ and ‘Standard unformulated lutein extract (Reference product)’. The dispersion profile is given in FIG. 2. The result shows that the ‘Test product’ dispersed easily without any mechanical stirring whereas the ‘Standard unformulated lutein extract’ was non-dispersible as it was floating on top of the water.

### Example 5: Bioavailable Astaxanthin Composition

| Sr. No. | Ingredients | Quantity |
|---|---|---|
| 1. | Asthaxanthin extract (Standardized) | 25 g |
| 2. | Fresh turmeric rhizome extract (on dry basis) | 75 g |
| | Total | 100 g |

### Example 6: Process for Preparation of Bioavailable Asthaxanthin Composition of Example 5

a) Washing 1000 g of fresh turmeric rhizome with RO water, slicing the rhizome, followed by extraction using low speed screw extruder with 0.5 mm to 1 mm mesh to separate the liquid extract and filtering the extract with 100-500 micron filter;
b) heating the fresh turmeric liquid extract obtained in step (a) for 30 minutes at 60° C. with continuous stirring using a homogenizer;
c) slowly adding 25 g of asthaxanthin extract to the liquid extract (75 g of fresh turmeric rhizome extract on dry basis) of step (b) with continuous stirring;
d) homogenising the mixture of fresh turmeric liquid extract and asthaxanthin extract of step (c) at 2000 RPM and 40° C. for 30 minutes to obtain viscous slurry; and
e) vacuum drying the slurry of step (d) at 60° C. for 8 hours using Rotary Vacuum Dryer (RVD); and
f) milling the flakes of step (e) to obtain free flowing powder (particle size>120 mesh) of self-dispersible Asthaxanthin composition.

Yield of Fresh turmeric liquid extract: 750 g

### Example 7: Bioavailable Lutein Composition

| Sr. No. | Ingredients | Quantity |
|---|---|---|
| 1. | Lutein extract (Standardized to 80%) | 25 g |
| 2. | Fresh beetroot extract (on dry basis) | 75 g |
| | Total | 100 g |

### Example 8: Process for Preparation of Bioavailable Lutein Composition of Example 7

a) Washing 1000 g of fresh taproots of beet with RO water followed by extraction using low speed screw extruder with 0.5 mm to 1 mm mesh to separate the liquid extract and filtering the extract with 100-500 micron filter;
b) heating the fresh beetroot liquid extract obtained in step (a) for 30 minutes at 40° C. with continuous stirring using a homogenizer;
c) slowly adding 25 g of lutein extract to the liquid extract (75 g of fresh beetroot extract on dry basis) of step (b) with continuous stirring;
d) homogenizing the mixture of fresh beetroot liquid extract and lutein extract of step (c) at 2000 RPM and 40° C. for 30 minutes to obtain viscous slurry;
e) vacuum drying the slurry of step (d) at 40° C. for 8 hours using Rotary Vacuum Dryer (RVD); and
f) milling the flakes of step (e) to obtain free flowing powder (particle size>120 mesh) of self-dispersible Lutein composition.

Yield of Fresh beetroot liquid extract: 750 g

### Example 9: Bioavailable Lutein Composition

| Sr. No. | Ingredients | Quantity |
|---|---|---|
| 1. | Lutein extract (Standardized to 80%) | 25 g |
| 2. | Fresh potato extract (on dry basis) | 75 g |
| | Total | 100 g |

### Example 10: Process for Preparation of Bioavailable Lutein Composition of Example 9

a) Washing 1500 g of fresh potato tubers with RO water followed by extraction using low speed screw extruder with 0.5 mm to 1 mm mesh to separate the liquid extract and filtering the extract with 100-500 micron filter;
b) heating the fresh potato liquid extract obtained in step (a) for 30 minutes at 40° C. with continuous stirring using a homogenizer;
c) slowly adding 25 g of lutein extract to the liquid extract (75 g of fresh potato extract on dry basis) of step (b) with continuous stirring;
d) homogenizing the mixture of fresh potato liquid extract and lutein extract of step (c) at 2000 RPM and 40° C. for 30 minutes to obtain viscous slurry;
e) vacuum drying the slurry of step (d) at 40° C. for 8 hours using Rotary Vacuum Dryer (RVD); and
f) milling the flakes of step (e) to obtain free flowing powder of self-dispersible Lutein composition.

Yield of Fresh potato liquid extract: 1000 g

### Example 11: Boswellia Extract Composition

| Sr. No. | Ingredients | Quantity |
|---|---|---|
| 1. | Boswellia extract (Standardized to 70% AKBA) | 32 g |
| 2. | Fresh turmeric rhizome extract (on dry basis) | 68 g |
| | Total | 100 g |

### Example 12: Process for Preparation of Boswellia Extract Composition of Example 11

a) Washing 1000 g of fresh turmeric rhizome with RO water, slicing the rhizome followed by extraction using low speed screw extruder with 0.5 mm to 1 mm mesh to separate the liquid extract and filtering the extract with 100-500 micron filter to obtain the bio-enhancing/self-dispersing agent;
b) heating the fresh turmeric liquid extract obtained in step (a) for 30 minutes at 40° C. with continuous stirring using a homogenizer;
c) slowly adding 32 g of Boswellia extract to the liquid extract (68 g of fresh turmeric rhizome extract on dry basis) of step (b) with continuous stirring;
d) homogenizing the mixture of step (c) at 2000 RPM for 30 minutes at 40° C. to obtain uniform viscous slurry;
e) vacuum drying the slurry of step (d) at 60° C. for 8 hours using Rotary Vacuum Dryer (RVD); and
f) milling the flakes of step (e) using communiting mill to obtain free flowing powder of self-dispersible Boswellia extract composition.

Yield of fresh turmeric liquid extract: 680 g

The AKBA content in the final product of Example 11 was 46.25%

### Example 13: Boswellia Extract Composition

| Sr. No. | Ingredients | Quantity |
|---|---|---|
| 1. | Boswellia extract (Standardized to 70%) | 75 g |
| 2. | Fresh turmeric rhizome extract (on dry basis) | 25 g |
| | Total | 100 g |

### Example 14: Process for Preparation of Boswellia Extract Composition of Example 13

a) Washing 300 g of fresh turmeric rhizome with RO water, slicing the rhizome followed by squeezing using low speed screw extruder with 0.5 mm to 1 mm mesh to separate the liquid extract, and filtering the extract with 100 micron filter to obtain the bio enhancing/self dispersing agent;
b) heating the fresh turmeric liquid extract obtained in step (a) for 30 minutes at 40° C. with continuous stirring using a homogenizer;
c) slowly adding 75 g of Boswellia extract to the liquid extract (25 g of fresh turmeric rhizome extract on dry basis) of step (b) with continuous stirring;
d) homogenizing the mixture of step (c) at 2000 RPM for 30 minutes at 40° C. to obtain uniform slurry;
e) vacuum drying the slurry of step (d) at 80° C. for 8 hours using Rotary Vacuum Dryer (RVD); and
f) milling the flakes by using communiting mill of step (e) to obtain free flowing powder (particle size>120 mesh) of self-dispersible Boswellia extract composition.

Yield of Fresh turmeric liquid extract: 250 g

Curcuminoid content of fresh turmeric liquid extract: <3%

### Example 15: Comparative Self-Dispersion Profile of ‘Boswellia Extract Composition of Example 13’ Vis-à-Vis ‘Standard Unformulated Boswellia Extract’

A comparative self-dispersion study was carried out between the ‘Composition of Example 13 (Test product)’ and ‘Standard unformulated Boswellia extract (Reference product)’. The dispersion profile is given in FIG. 3. The result shows that the ‘Test product’ dispersed easily without any mechanical stirring where as the ‘Standard unformulated Boswellia extract’ was non-dispersible as it was floating on top of the water.

### Example 16: Pepper Extract Composition

| Sr. No. | Ingredients | Quantity |
|---|---|---|
| 1. | Pepper extract (Standardized to 95%) | 85 g |
| 2. | Fresh turmeric rhizome extract (on dry basis) | 15 g |
| | Total | 100 g |

### Example 17: Process for Preparation of Pepper Extract Composition of Example 16

a) Washing 250 g of fresh turmeric rhizome with RO water, slicing the rhizome, followed by extraction using low speed screw extruder with 0.5 mm to 1 mm mesh to separate the liquid extract, and filtering the extract with 100-500 micron filter to obtain the bio-enhancing/self dispersing agent;
b) heating the fresh turmeric liquid extract obtained in step (a) for 30 minutes at 40° C. with continuous stirring using a homogenizer;
c) slowly adding 85 g of pepper extract (95% of piperine) to the liquid extract (15 g of fresh turmeric rhizome extract on dry basis) of step (b) with continuous stirring;
d) homogenizing the mixture of step (c) at 2000 RPM for 30 minutes at 40° C. to obtain viscous uniform slurry;
e) vacuum drying the slurry of step (d) at 80° C. for 8 hours using Rotary Vacuum Dryer (RVD); and
f) milling the flakes by using communiting mill of step (e) to obtain free flowing powder (particle size>120 mesh) of self-dispersible Pepper extract composition.

Yield of fresh turmeric liquid extract: 200 g

### Example 18: Release Profile of ‘Pepper Extract Composition of Example 16’ Vs. ‘Standard Unformulated Pepper Extract (95% Piperine)’

Solubility of piperine from ‘Pepper extract composition of Example 16 (test composition)’ was done in phosphate buffer 6.8 in comparison with standard unformulated pepper extract (95% Piperine). 250 mg each of each sample was added into 400 ml of 6.8 buffer in separate beakers kept in water bath at the temperature of 37° C. with continuous stirring at 50 rpm. Samples were collected at regular intervals of 0, 1, 2, 3, 4 and 5 hrs and filtered using 0.22 m micro filter. The absorbance of the read at 343 nm using UV-Spectrophotometer. Absorbance over time graph is given in FIG. 4. Increased absorbance corresponds to the increased solubility of piperine from the composition. This test clearly proves that the test composition has superior solubility over unformulated extract.

### Example 19: Comparative Self-Dispersion Profile of ‘Pepper Extract Composition of Example 16’ Vis-à-Vis ‘Standard Unformulated Pepper Extract’

A comparative self-dispersion study was carried out between the ‘Composition of Example 16 (Test product)’ and ‘Standard unformulated Pepper extract (Reference product)’. The dispersion profile is given in FIG. 5. The result shows that the ‘Test product’ dispersed easily without any mechanical stirring whereas the ‘Standard unformulated Pepper extract’ was non-dispersible as it was floating on top of the water.

### Example 20: Pepper Extract Composition

| Sr. No. | Ingredients | Quantity |
|---|---|---|
| 1. | Pepper extract (Standardized to 95% ) | 80 g |
| 2. | Fresh turmeric rhizome extract (on dry basis) | 20 g |
| | Total | 100 g |

### Example 21: Process for Preparation of Pepper Extract Composition of Example 20

a) Washing 300 g of fresh turmeric rhizome with RO water, followed by extraction using low speed screw extruder with 0.5 mm to 1 mm mesh to separate the liquid extract and filtering the extract with 100-500 micron filter to obtain the bio-enhancing/self-dispersing agent;
b) heating the fresh turmeric liquid extract obtained in step (a) for 30 minutes at 40° C. with continuous stirring using a homogenizer;
c) slowly adding 80 g of pepper extract (95% of piperine) to the liquid extract (20 g of fresh turmeric rhizome extract) of step (b) with continuous stirring;
d) homogenizing the mixture of step (c) at 2000 RPM for 30 minutes at 40° C. to obtain viscous uniform slurry;
e) vacuum drying the slurry of step (d) at 80° C. for 8 hours using Rotary Vacuum Dryer (RVD); and
f) milling the flakes by using communiting mill of step (e) to obtain free flowing powder (particle size>120 mesh) of self-dispersible Pepper extract composition.

Yield of fresh turmeric liquid extract: 200 g

### Example 22: Coenzyme Q10 Composition

| Sr. No. | Ingredients | Quantity |
|---|---|---|
| 1. | Coenzyme Q10 (Standardized to 99%) | 23 g |
| 2. | Ascorbic acid | 4 g |
| 3. | Fresh turmeric rhizome extract (on dry basis) | 73 g |
| | Total | 100 g |

### Example 23: Process for Preparation of Coenzyme Q10 Composition of Example 22

a) Washing 1000 g of fresh turmeric rhizome with RO water followed by extraction using low speed screw extruder with 0.5 mm to 1 mm mesh to separate the liquid extract and filtering the extract with 100-500 micron filter to obtain the bio-enhancing/self-dispersing agent;
b) heating the fresh turmeric liquid extract obtained in step (a) for 30 minutes at 60° C. with continuous stirring using a homogenizer;
c) adding 23 g of Coenzyme Q10 (standardized to 99%) to the liquid extract (73 g of fresh turmeric rhizome extract on dry basis) of step (b) followed by adding 4 g of ascorbic acid with continuous stirring;
d) homogenizing the mixture of step (c) at 2000 RPM for 30 minutes at 40° C. to obtain viscous uniform slurry;
e) vacuum drying the slurry of step (d) at 50° C. for 8 hours using Rotary Vacuum Dryer (RVD); and
f) milling the flakes by using communiting mill of step (e) to obtain free flowing powder of self-dispersible Coenzyme Q10 composition.

Yield of fresh turmeric extract liquid: 730 g

### Example 24: Comparative Self-Dispersion Profile of ‘Coenzyme Q10 Composition of Example 22’ Vis-à-Vis ‘Standard Unformulated Coenzyme Q10 Extract’

A comparative self dispersion study was carried out between the ‘Composition of Example 22 (Test product)’ and ‘Standard unformulated Coenzyme Q10 Extract (Reference product)’. The dispersion profile is given in FIG. 6. The result shows that the ‘Test product’ dispersed easily without any mechanical stirring whereas the ‘Standard unformulated Coenzyme Q10 extract’ was non-dispersible as it was floating on top of the water.

### Example 25: Palmitoylethanolamide Composition

| Sr. No. | Ingredients | Quantity |
|---|---|---|
| 1. | Palmitoylethanolamide | 85 g |
| 2. | Fresh turmeric rhizome extract (on dry basis) | 15 g |
| | Total | 100 g |

### Example 26: Process for Preparation of Palmitoylethanolamide Composition of Example 25

a) Washing 200 g of fresh turmeric rhizome with RO water followed by extraction using low speed screw extruder with 0.5 mm to 1 mm mesh to separate the liquid extract, and filtering the extract with 100-500 micron filter to obtain the bio-enhancing/self dispersing agent;
b) heating the fresh turmeric liquid extract obtained in step (a) for 30 minutes at 60° C. with continuous stirring using a homogenizer;
c) adding 85 g of palmitoylethanolamide to the liquid extract (15 g of fresh turmeric rhizome extract on dry basis) of step (b) with continuous stirring;
d) homogenizing the mixture of step (c) at 2000 RPM for 30 minutes at 40° C. to obtain viscous uniform slurry;
e) vacuum drying the slurry of step (d) at 60° C. for 8 hours using Rotary Vacuum Dryer (RVD); and
f) milling the flakes by using communiting mill of step (e) to obtain free flowing powder (particle size>120 mesh) of self-dispersible palmitoylethanolamide composition.

Yield of fresh turmeric liquid extract: 150 g

### Example 27: Comparative Self-Dispersion Profile of ‘Palmitoylethanolamide Composition of Example 25’ Vis-à-Vis ‘Standard Unformulated Palmitoylethanolamide’

A comparative self dispersion study was carried out between the ‘Composition of Example 25 (Test product)’ and ‘Standard unformulated Palmitoylethanolamide (Reference product)’. The dispersion profile is given in FIG. 7A (test product) and FIG. 7B (reference product). The result shows that the ‘Test product’ dispersed easily without any mechanical stirring whereas the ‘Standard unformulated Coenzyme Q10 extract’ was non-dispersible as it was floating on top of the water.

### Example 28: Berberine Extract Composition

| Sr. No. | Ingredients | Quantity |
|---|---|---|
| 1. | Berberine extract (Standardized.) | 50 g |
| 2. | Fresh turmeric rhizome extract (on dry basis) | 50 g |
| | Total | 100 g |

### Example 29: Process for Preparation of Berberine Extract Composition of Example 28

a) Washing 600 g of fresh turmeric rhizome with RO water followed by extraction using low speed screw extruder with 0.5 mm to 1 mm mesh to separate the liquid extract and filtering the extract with 100-500 micron filter to obtain the bio-enhancing/self-dispersing agent;
b) heating the fresh turmeric liquid extract obtained in step (a) for 30 minutes at 60° C. with continuous stirring using a homogenizer;
c) slowly adding 50 g of berberine extract to the liquid extract (50 g of fresh turmeric rhizome extract) of step (b) with continuous stirring;
d) homogenizing the mixture of step (c) at 2000 RPM for 30 minutes at 40° C. to obtain viscous uniform slurry; and
e) vacuum drying the slurry of step (d) at 40° C. for 8 hours using Rotary Vacuum Dryer (RVD); and
f) milling the flakes by using communiting mill of step (e) to obtain free flowing powder of self-dispersible berberine extract composition.

Yield of fresh turmeric liquid extract: 500 g

### Example 30: Vitamin D Composition

| Sr. No. | Ingredients | Quantity |
|---|---|---|
| 1. | Vitamin D | 80 g |
| 2. | Fresh turmeric rhizome extract (on dry basis) | 20 g |
| | Total | 100 g |

### Example 31: Process for Preparation of Vitamin D Composition of Example 30

a) Washing 300 g of fresh turmeric rhizome with RO water, slicing the rhizome followed by extraction using low speed screw extruder with 0.5 mm to 1 mm mesh to separate the liquid extract, and filtering the extract with 100-500 micron filter to obtain the bio-enhancing/self dispersing agent;
b) heating the fresh turmeric liquid extract obtained in step (a) for 30 minutes at 60° C. with continuous stirring using a homogenizer;
c) adding 80 g Vitamin D to the liquid extract (20 g of fresh turmeric rhizome extract on dry basis) of step (b) with continuous stirring;
d) homogenizing the mixture of step (c) at 2000 RPM for 30 minutes at 40° C. to obtain viscous uniform slurry;
e) vacuum drying the slurry of step (d) at 60° C. for 8 hours using Rotary Vacuum Dryer (RVD); and
f) milling the flakes by using communiting mill of step (e) to obtain free flowing powder of self-dispersible Vitamin D composition.

Yield of fresh turmeric liquid extract: 200 g

### Example 32: Bioavailable Lutein Composition

| Sr. No. | Ingredients | Quantity |
|---|---|---|
| 1. | Lutein extract (Standardized to 80%) | 5 g |
| 2. | Fresh turmeric rhizome extract (on dry basis) | 95 g |
| | Total | 100 g |

### Example 33: Process for Preparation of Bioavailable Lutein Composition of Example 32

a) Washing 1500 g of Fresh turmeric rhizome with RO water, slicing the rhizome followed by extraction using low speed screw extruder with 0.5 mm to 1 mm mesh to separate the liquid extract and filtering the extract with 100-500 micron filter;
b) heating the Fresh turmeric rhizome extract obtained in step (a) for 30 minutes at 40° C. with continuous stirring using a homogenizer;
c) slowly adding 5 g of lutein extract to the liquid extract (95 g of Fresh turmeric rhizome extract on dry basis) of step (b) with continuous stirring;
d) homogenizing the mixture of Fresh turmeric rhizome liquid extract and lutein extract of step (c) at 2000 RPM and 40° C. for 30 minutes to obtain viscous slurry;
e) vacuum drying the slurry of step (d) at 40° C. for 8 hours using Rotary Vacuum Dryer (RVD); and
f) milling the flakes of step (e) to obtain free flowing powder of self-dispersible Lutein composition.

Yield of Fresh turmeric rhizome extract: 1000 g

### Example 34: Bioavailable S-Allyl-Cysteine (SAC) Composition

| Sr. No. | Ingredients | Quantity |
|---|---|---|
| 1. | Aged black garlic extract (Standardized to SAC) | 25 g |
| 2. | Fresh Allium sativum bulb extract (on dry basis) | 75 g |
| | Total | 100 g |

### Example 35: Process for Preparation of Bioavailable S-Allyl-Cysteine (SAC) Composition of Example 34

a) Removing the outer layer of 1500 g of Fresh Allium sativum bulb, washing with RO water followed by extraction using low speed screw extruder with 0.5 mm to 1 mm mesh to separate the liquid extract and filtering the extract with 100-500 micron filter;
b) heating the Fresh Allium sativum bulb extract obtained in step (a) for 120 minutes at 80° C. with continuous stirring using a homogenizer;
c) slowly adding 25 g of Aged black garlic extract to the liquid extract (75 g of Allium sativum bulb extract on dry basis) of step (b) with continuous stirring;
d) homogenizing the mixture of fresh potato liquid extract and Aged black garlic extract of step (c) at 2000 RPM and 40° C. for 30 minutes to obtain viscous slurry;
e) vacuum drying the slurry of step (d) at 40° C. for 8 hours using Rotary Vacuum Dryer (RVD); and
f) milling the flakes of step (e) to obtain free flowing powder of self-dispersible S-allyl-cysteine (SAC) composition.

Yield of Fresh Allium sativum bulb extract: 900 g

The SAC content in the final product of Example 33 was 0.81%

### Example 36: Bioavailable Boswellia serrata Extract Composition

| Sr. No. | Ingredients | Quantity |
|---|---|---|
| 1. | Boswellia serrata extract (Standardized to 70%) | 75 g |
| 2. | Fresh aloe vera extract (on dry basis) | 25 g |
| | Total | 100 g |

### Example 37: Process for Preparation of Boswellia serrata Extract Composition of Example 36

g) Removing the outer green layer of 1000 g of Fresh Aloe vera leaves, washing with RO water followed by extraction using low speed screw extruder with 0.5 mm to 1 mm mesh to separate the liquid extract and filtering the extract with 100-500 micron filter;
h) heating the Fresh Aloe vera leaf extract obtained in step (a) for 30 minutes at 50° C. with continuous stirring using a homogenizer;
i) slowly adding 25 g of Boswellia serrata extract to the liquid extract (25 g of Fresh Aloe vera leaf extract on dry basis) of step (b) with continuous stirring;
j) homogenizing the mixture of Fresh Aloe vera leaf liquid extract and Boswellia serrata extract of step (c) at 2000 RPM and 40° C. for 30 minutes to obtain viscous slurry;
k) vacuum drying the slurry of step (d) at 40° C. for 8 hours using Rotary Vacuum Dryer (RVD); and
l) milling the flakes of step (e) to obtain free flowing powder of Boswellia serrata extract composition.

Yield of Fresh Aloe vera leaf extract: 600 g

The AKBA content in the final product of Example 36 was 43.23%

### Example 38: Bioavailable Silybum marianum Extract Composition

| Sr. No. | Ingredients | Quantity |
|---|---|---|
| 1. | Silybum marianum extract | 75 g |
| 2. | Fresh aloe vera extract (on dry basis) | 25 g |
| | Total | 100 g |

### Example 39: Process for Preparation of Silybum marianum Extract Composition of Example 38

a) Washing 1000 g of Fresh turmeric rhizome with RO water, slicing the rhizome followed by extraction using low speed screw extruder with 0.5 mm to 1 mm mesh to separate the liquid extract and filtering the extract with 100-500 micron filter;
b) heating the Fresh turmeric rhizome extract obtained in step (a) for 30 minutes at 40° C. with continuous stirring using a homogenizer;
c) slowly adding 75 g of Silybum marianum extract to the liquid extract (25 g of Fresh turmeric rhizome extract on dry basis) of step (b) with continuous stirring;
d) homogenizing the mixture of Fresh turmeric rhizome liquid extract and Silybum marianum extract of step (c) at 2000 RPM and 40° C. for 30 minutes to obtain viscous slurry;
e) vacuum drying the slurry of step (d) at 40° C. for 8 hours using Rotary Vacuum Dryer (RVD); and
f) milling the flakes of step (e) to obtain free flowing powder of self-dispersible Silybum marianum extract composition.

Yield of Fresh turmeric rhizome extract: 700 g

We claim:

1. A composition for enhancing the bioavailability of phytochemicals, comprising:
a) a phytochemical selected from the group consisting of carotenoids, stilbenoids, isoflavones, terpenes, isoquinoline alkaloids, phenolic compounds, saponins, flavonoids, quinones, fatty acids, derivatives thereof, and mixtures thereof; and
b) an extract of a fresh plant part such as fruits, seeds, leaves, and rhizomes selected from the group consisting of Curcuma longa, Zingiber officinale, berry fruits, Beta vulgaris, Solanum tuberosum, Aloe barbadensis, Daucus carota, Mangifera indica, Manihot esculenta, Cocos nucifera, (Coconut), Allium sativum bulb, Siraitia grosvenorii (monk fruit) and mixtures thereof;
wherein the extract of the fresh plant part is a self-dispersing agent;
wherein the self-dispersing agent enhances the bioavailability of the phytochemical.

2. The composition as claimed in claim 1, wherein the extract of the fresh plant part is prepared by slow extrusion of a fresh plant part to obtain a liquid extract.

3. The composition as claimed in claim 1, wherein the fresh plant part contains at least 40% by weight of water.

4. The composition as claimed in claim 1, wherein the phytochemical is a lutein, astaxanthin, piperine, cannabinoids, boswellic acid, coenzyme Q10, palmitoylethanolamide, S-allyl-cysteine, Silymarin, berberine, vitamin K7, Spearmint extract and vitamin D and mixtures thereof.

5. The composition as claimed in claim 1, wherein the extract of the fresh plant part is selected from the group consisting of extracts of rhizomes of Curcuma longa, taproots of Beta vulgaris, tubers of Solanum tuberosum, leaves of Aloe barbadensis, taproots of Daucus carota, fruits of Mangifera indica, roots of Manihot esculenta, fruits of Cocos nucifera, Allium sativum bulb, Siraitia grosvenorii (monk fruit), and mixtures thereof.

6. A composition for enhancing the bioavailability of phytochemicals, comprising:
a) a phytochemical; and
b) an extract of a fresh plant part selected from the group consisting of Curcuma longa, Beta vulgaris, Solanum tuberosum, Aloe barbadensis, Daucus carota, Mangifera indica, Manihot esculenta, Cocos nucifera, Allium sativum bulb, Siraitia grosvenorii (monk fruit) and mixtures thereof;
wherein the extract of the fresh plant part is a self-dispersing agent;
wherein the self-dispersing agent enhances the bioavailability of the phytochemical;
wherein the phytochemical is selected from the group consisting of Lutein, Zeaxanthin, Lycopene, astaxanthin, beta-carotene, berberine, resveratrol; genistein; coenzyme Q10, palmitoylethanolamide, piperine, a cannabinoid, a boswellic acids, a chlorogenic acid, silymarin, silibinin, catechin, a gingerol, a shogaol, an ellagic acid, quercetin, caffeine, a caffeic acid derivative, iron, calcium, an ecdysteroid, ecdysterone, a rosavin, salidroside, a curculigoside, an omega-3-fatty acid, echinasea, a gymneric acid, S-allyl-cysteine, melatonin, S-allyl-cysteine, citrus bioflavonoids, forskolin, huperzine, hypericin, hyperforin, a phytoestrogen, a ginsenoside, valerenic acid, vitamin D, vitamin E, menthol, theacrine, and mixtures thereof.

7. The composition as claimed in claim 6, wherein the phytochemical is selected from the group consisting of lutein, astaxanthin, piperine, a cannabinoid, a boswellic acid, coenzyme Q10, S-allyl-cysteine, palmitoylethanolamide, silymarin, berberine, vitamin k7 and vitamin D.

8. The composition as claimed in claim 6, wherein the phytochemical is selected from the group consisting of lutein, piperine, a boswellic acid, and coenzyme Q10.

9. The composition as claimed in claim 6, wherein the extract of the fresh plant part is selected from the group consisting of rhizomes of Curcuma longa, taproots of Beta vulgaris, tubers of Solanum tuberosum, leaves of Aloe barbadensis, taproots of Daucus carota, fruits of Mangifera indica, roots of Manihot esculenta, fruits of Cocos nucifera, bulb of Allium sativum, fruit of Siraitia grosvenorii and mixtures thereof.

10. The composition as claimed in claim 9, wherein the extract of the fresh plant part is selected from the group consisting of rhizomes of Curcuma longa, taproots of Beta vulgaris, leaves of Aloe barbadensis, tubers of Solanum tuberosum, bulb of Allium sativum, and mixtures thereof.

11. The composition as claimed in claim 1, wherein the phytochemical is present in an amount ranging from 5% to 95%; and
the extract of the fresh plant part is present in an amount ranging from 5% to 95%;
based on the combined dry weight of the phytochemical and the extract of the fresh plant part.

12. The composition as claimed in claim 6, wherein the phytochemical is present in an amount ranging from 5% to 95%; and
the extract of the fresh plant part is present in an amount ranging from 5% to 95%;
based on the combined dry weight of the phytochemical and the extract of the fresh plant part.

13. The composition as claimed in claim 1, wherein the phytochemical is present in an amount ranging from 5% to 85%; and
the extract of the fresh plant part is present in an amount ranging from 15% to 95%;
based on the combined dry weight of the phytochemical and the extract of the fresh plant part.

14. The composition as claimed in claim 6, wherein the phytochemical is present in an amount ranging from 25% to 85%; and
the extract of the fresh plant part is present in an amount ranging from 15% to 75%;
based on the combined dry weight of the phytochemical and the extract of the fresh plant part.

15. The composition as claimed in claim 1, wherein the composition is formulated into a dosage forms selected from the group consisting of tablets, capsules, pills, solutions, pastes, lozenges, ready to drink beverages (RTD), beverages, fortified food, chocolates, instant mixes, and beverages.

16. The composition as claimed in claim 6, wherein the composition is formulated into a dosage forms selected from the group consisting of tablets, capsules, pills, solutions, pastes, lozenges, ready to drink beverages (RTD), beverages, fortified food, chocolates, instant mixes, and beverages.

17. A process for preparing a self-dispersible composition for enhancing the bioavailability of a phytochemical, comprising:
a) washing and slicing a fresh plant part into small pieces and extracting the small pieces of the fresh plant part using a low RPM screw extruder with 0.5 mm to 1 mm mesh/filter press to obtain an extract of the fresh plant part;
wherein the fresh plant part is selected from the group consisting of rhizomes of Curcuma longa, taproots of Beta vulgaris, tubers of Solanum tuberosum, leaves of Aloe barbadensis, taproots of Daucus carota, fruits of Mangifera indica, roots of Manihot esculenta, fruits of Cocos nucifera, bulb of Allium sativum, fruit of Siraitia grosvenorii and mixtures thereof;
b) filtering the extract of the fresh plant part of step (a) using a 100-500 micron filter to remove sedimentable particles, followed by heating the filtered extract for 30 to 120 minutes at 30° C. to 80° C. with continuous stirring;
c) adding a phytochemical to the extract of step (b) to obtain a mixture;
wherein the phytochemical is selected from the group consisting of carotenoids, stilbenoids, isoflavones, terpenes, isoquinoline alkaloids, phenolic compounds, saponins, flavonoids, quinones, fatty acids, derivatives thereof, and mixtures thereof and
d) homogenizing the mixture of step (c) at 200 to 10000 RPM for 30 to 60 minutes at 25° C. to 80° C., followed by drying the homogenized mixture; and
e) powdering the homogenized mixture to obtain a free-flowing powder of the self-dispersible composition.

18. The method of claim 17, wherein the fresh plant part contains at least 40% by weight of water.

19. A method of treating inflammatory disease, respiratory diseases, heart diseases, sexual diseases, vascular disease, liver disease, cognitive diseases, eye diseases, skin disease, anti-aging and stress by administering therapeutically effective amount of composition as claimed in claim 1 to a subject in need thereof.

20. A method of treating inflammatory disease, respiratory diseases, heart diseases, sexual diseases, vascular disease, liver disease, cognitive diseases, eye diseases, skin disease and stress by administering therapeutically effective amount of composition as claimed in claim 6 to a subject in need thereof.
